(19) United States
(12) Patent Application Publication
(10) Pub. No.: US 2005/0148947 A1
(43) Pub. Date: Jul. 7, 2005

(54) Cannula locking device

(75) Inventor: Kenneth E. Kadziauskas, Coto de Caza, CA (US)

Correspondence Address: Pillsbury Winthrop LLP, Suite 2800, 725 S. Figueroa Street, Los Angeles, CA, 90017, US

(73) Assignee: ADVANCED MEDICAL OPTICS, INC.

(21) Appl. No.: 10/748,101

(22) Filed: Dec. 30, 2003

(51) Int. Cl. A61M 5/00

(52) U.S. Cl. 604/240

(57) ABSTRACT

A cannula hood for preventing disconnection of a cannula assembly from a syringe apparatus is provided. The cannula hood comprises a cylindrical portion and a tapered portion. The cylindrical portion may be designed to fit over a syringe body and to be retained by a friction fit. The cannula hood may adapt a single compartment syringe or a multi-compartment syringe.

### FIELD OF THE INVENTION

The present invention relates to syringes. In particular, the invention relates to a means for securing a cannula assembly to a syringe body. Such securing means is particularly useful, for example, when such syringe is used with viscoelastic agents in ophthalmic surgery.

### BACKGROUND OF THE INVENTION

The term viscosurgery refers to the use of viscoelastic substances in surgery. Ophthalmic viscosurgery encompasses a broad range of intraocular surgical procedures including, for example, cataract surgery, cornea surgery, glaucoma surgery, vitreous surgery, trauma surgery, intraocular lens implantation and retinal detachment surgery. Ophthalmic viscosurgical procedures frequently involve forming an incision in the cornea and subsequently gaining instrument access to one or all of the anterior chamber, lens capsular bag and posterior chamber for the purpose of manipulating target eye structures.

During ophthalmic viscosurgery, viscoelastic solutions are often introduced into the eye to protect tissues and to ease both the introduction of instruments into the surgical field and the concomitant manipulation of eye tissues. Some of the tasks performed by viscoelastics during ophthalmic surgery include lubrication, the coating of surfaces, the creation and maintenance of eye chamber spaces, the separation of tissues, the displacement of mobile tissue, the blocking of outflow ocular fluids, the confinement of bleeding, the exclusion of inflammatory materials and the dampening of instrument movement in the eye.

Cataract surgery is a routine ophthalmic viscosurgical procedure. The procedure can be divided into several steps. Following pupil dilation and local anesthesia, an incision is made through the cornea into the anterior chamber of the eye. Once the anterior chamber is punctured, the natural aqueous humor leaks out and the chamber loses its characteristic shape and depth. A viscoelastic product is then introduced into the anterior chamber so that the chamber regains its former volume and so that vulnerable tissues such as the corneal endothelium are protected. Next, a capsulotomy is performed, which involves making a hole in the anterior portion of the lens capsular bag. Extraction of the lens itself is then most often accomplished by phacoemulsification (disintegration of the lens by ultrasound, followed by aspiration of the resultant lens particles) or by extracapsular cataract extraction (removal of the lens in one piece). At this stage, a viscoelastic product is again needed to maintain the natural volume of the capsular bag and to facilitate the introduction of a synthetic lens. Upon completion of the surgery, the viscoelastic compound is removed from the eye and replaced with a physiologically compatible salt solution. Generally, viscoelastics cannot be left in the eye due to the compounds' viscosity and the eye's resultant inability to process the compounds through its filtering system. Leftover viscoelastics that cannot be adequately processed by the eye cause a detrimental increase in intraocular pressure.

Viscoelastics are solutions that have viscous, elastic and pseudoplastic properties. Viscoelastics, by definition, have dual properties. They act as both viscous liquids and as elastic solids or gels. The typical viscoelastic is an aqueous solution containing a polysaccharide such as sodium hyaluronate, chondroitin sulfate or hydroxypropylmethylcellulose. Concentrations of viscoelastics vary from 10-70 mg/ml and molecular masses (expressed as mass average relative molecular masses) vary from 20,000 daltons (chondroitin sulfate) to 5,000,000 (sodium hyaluronate).

There are two main classes of viscoelastic compounds: dispersive and cohesive. Viscoat™ (Alcon Laboratories, Inc., Forth Worth, Tex.) is an example of a dispersive compound while Healon™ (Pharmacia, Inc., Piscataway, N.J.) is an example of a cohesive compound. Dispersive products are typically high concentration (20 mg/ml to 70 mg/mL) solutions of low molecular weight (less than 500,000 daltons) polysaccharides. Cohesive products are typically low concentration (10-20 mg/mL) solutions of high molecular weight (1 to 5 million daltons) polysaccharides.

During an ophthalmic viscosurgical procedure such as cataract surgery, viscoelastics are typically introduced into the eye through a relatively narrow-gauge cannula coupled to a syringe. The small cannula size is preferred because of the need to use instruments of the smallest possible size when operating in the eye. However, use of a small cannula presents two related difficulties, both stemming from the high viscosity of the most commonly used viscoelastics. First, it is difficult for the operator of the syringe to generate sufficient force to extrude the viscoelastic solution from the syringe into the cannula and, ultimately, out of the cannula. Second, and importantly for present purposes, the pressure built up at the syringe/cannula junction during extrusion of the viscoelastic solution can be exceedingly high, potentially resulting in the cannula becoming disconnected from the syringe.

Commonly assigned U.S. Pat. Nos. 5,419,775 and 5,554,133 to Haffner et al. disclose a syringe apparatus that allows the operator to generate sufficient force to extrude a viscoelastic solution. Disclosed in the Haffner patents is an adapter that attaches to a syringe body and provides an operator with an enlarged grip. The enlarged grip allows the operator to depress a plunger of the syringe with a greater degree of opposing force between the operator's thumb and forefinger than would be possible in the case of a conventional syringe apparatus.

However, the difficulty presented by excess pressure at the syringe/cannula junction has been more difficult to solve. Although it may serve to ameliorate the excess pressure, employing a cannula with a larger diameter is undesirable for ophthalmic applications for the reasons stated above. Thus, various attempts have been made to provide a backup to the conventional Luer lock fitting that is commonly used to couple a cannula to a syringe.

U.S. Pat. No. 5,925,032 to Clements discloses a syringe assembly comprising a syringe body, a cannula assembly and a pair of outer sleeve halves. The cannula assembly is disposed upon the syringe body and the sleeve halves are slid over the cannula assembly and subsequently held closed by a retaining nut. It is disclosed that the resulting closed sleeve would restrict the movement of the cannula assembly and prevent the cannula assembly from becoming a projectile inside a patient's eye during surgery, even in the event that the cannula assembly became disconnected from the syringe body.

U.S. Pat. No. 5,876,379 to Beauvais et al. discloses a syringe assembly including a sleeve with an open flared end and an end having external and internal threads, a retaining nut with an internal thread designed to engage the external thread of the sleeve, and a cannula assembly having an external thread designed to engage the internal thread of the sleeve. The sleeve is designed to enclose a conventional syringe barrel, whereupon the cannula assembly is threaded onto the sleeve. Finally, the retaining nut is threaded onto the sleeve, thus restricting the movement of the cannula in the event that it becomes disconnected from the syringe body.

There are several disadvantages to the apparatuses disclosed in both the Clements and Beauvais patents. Both require significant additional components to be added to a conventional syringe assembly, including a sleeve and a retaining nut. The requirement for extra, relatively complex components generally increases the cost of manufacturing and, thus, the cost to the end user. In addition, viscoelastic solutions are commonly shipped to practitioners in pre-filled syringe apparatuses that are not fully assembled. Typically, the syringe body and cannula assembly are not yet joined and the end of the syringe body that is designed to accommodate the cannula assembly is plugged by some form of rubber stopper. Before use during ophthalmic surgery, the doctor or nurse must remove the stopper, attach the cannula assembly to the syringe body and, in the case of the Clements and Beauvais apparatuses, attach the sleeve and retaining nut to complete the syringe apparatus. Standing in direct conflict with the fact that time and simplicity are of the essence during ophthalmic viscosurgical procedures, these extra assembly steps may be time consuming and complex. Extra assembly steps are particularly undesirable from a practitioner's standpoint if additional syringe apparatuses must be assembled during surgery due to unforeseen circumstances that arise during ophthalmic viscosurgery from time to time.

Thus, the prior art does not achieve the most desirable outcome of providing a means for securely fixing a cannula assembly to a syringe body that does not involve excessive manufacturing expense and complexity of operation from the perspective of the end user. The prior art does not suggest a means for securing a cannula assembly to a syringe body that requires only minimal modifications to a commercially available syringe and can be quickly and easily used by a practitioner. Therefore, an inexpensive and easily used means for preventing harmful disconnections of cannula assemblies from syringe bodies is desired.

### BRIEF DESCRIPTION OF THE DRAWINGS

FIG. 1 shows a perspective view of a cannula locking device according to an embodiment of the present invention.

FIG. 2 shows a perspective view of a partially assembled single compartment syringe, according to an embodiment of the present invention.

FIG. 3 shows a partial axial sectional view of a single compartment syringe with a cannula attached, according to an embodiment of the present invention.

FIG. 4 shows an axial sectional view of a single compartment syringe, according to an embodiment of the present invention.

FIG. 5 shows an axial sectional view of an adapter for a single compartment syringe, according to an embodiment of the present invention.

FIG. 6 shows a perspective view of a cannula locking device of the present invention attached to single compartment syringe assembly, according to an embodiment of the present invention. In the areas of overlap between the cannula locking device and syringe assembly, the cannula locking device is drawn in sectional form.

FIG. 7 shows a perspective view of a bi-compartment syringe, according to an embodiment of the present invention.

FIG. 8 shows a perspective view of a cannula locking device of the present invention attached to a bi-compartment syringe assembly, according to an embodiment of the present invention. In the areas of overlap between the cannula locking device and bi-compartment syringe assembly, the cannula locking device is drawn in sectional form.

### DETAILED DESCRIPTION OF THE INVENTION

It has been discovered that a cannula assembly can best be secured to a syringe body through the use of a cannula hood according to the present invention. According to an embodiment of the present invention, the cannula hood may form a lumen and may form openings at its proximal and distal ends. Additionally, the cannula hood may comprise a cylindrical portion closest to its proximal end and a tapered portion closest to its distal end. The tapered portion may comprise a proximal section, a mid section and a distal section. The cylindrical portion may be designed to fit over the syringe body and the tapered portion may be designed to narrow at its apex to provide an opening that accommodates the diameter of a needle portion of the cannula assembly.

The cannula hood may be constructed of plastic or rubber materials such as, but not in any way limited to, silicone, latex-free rubber, soft PVC, polycarbonate or acrylic. The cannula hood may be uniform in its construction or, in the alternative, the cylindrical portion may be constructed of a softer material and the tapered portion may be constructed of a firmer material. By way of example only, the cylindrical portion may be constructed of silicone and the tapered portion may be constructed of PVC.

The cannula hood may adapt a conventional syringe apparatus designed for ophthalmic viscosurgery in a relatively simple and cost-effective manner that prevents dangerous disconnections of cannula assemblies from syringe bodies. Accordingly, a commercially available syringe body may be purchased along with a commercially available cannula assembly. The syringe body may be pre-filled with a viscoelastic solution and the pre-filled syringe body may be shipped to an end user along with the cannula assembly and a cannula hood according to the present invention. The end user, typically a doctor or nurse, may attach the cannula assembly to the syringe body at a conventional Luer lock fitting and then may slide the cannula hood over the needle portion of the cannula assembly and fix the cannula hood to the syringe body by way of a friction fit. One skilled in the art will realize that alternate means of attaching the cannula hood to the syringe are possible including, but not in any way limited to, clipping the cylindrical portion of the cannula hood to a Luer lock mechanism of a conventional syringe apparatus. One skilled in the art will also realize that the cannula hood of the present invention may be used with any commercially available filled or empty syringe body.

The cannula assembly may include a hub portion that facilitates coupling of the cannula assembly to the syringe body. The cannula hood may be designed such that the proximal section of the tapered portion has a diameter greater than the diameter of the hub portion, the mid section of the tapered portion has a diameter that is equal to the diameter of the hub portion and the distal section of the tapered portion has a diameter that is less than the diameter of the hub portion. If the cannula assembly were to become disconnected from the syringe body during a viscosurgical procedure, the hub portion of the cannula assembly would make contact with the tapered portion of the cannula hood and would be prevented from moving in a distal direction to a point beyond the mid section of the tapered portion of the cannula hood. The cannula assembly would therefore be prevented from making excessive movement towards the distal end of the cannula hood and, thus, would not become a projectile in the surgical field. Therefore, harmful disconnection of the cannula assembly from the syringe body would be prevented and damage to tissues in the surgical field would be ameliorated.

The cannula hood of the present invention may be attached to conventional single compartment syringe assemblies or to multi-compartment syringe assemblies. Various embodiments of the cannula hood of the present invention may also be designed in a multitude of sizes so as to accommodate syringe bodies and cannula assemblies of various sizes.

The cannula hood of the present invention has significant advantages over prior art devices for preventing harmful disconnections of cannula assemblies from syringe bodies. The present cannula hood employs a simple design and is constructed of common, inexpensive materials. Thus, manufacturing costs and corresponding end user costs will be lower than those associated with the prior art devices. In addition, the cannula hood disclosed herein can be used as a simple modification to commercially available syringes. Prior art devices require more extensive modifications to commercially available syringes, including the addition of sleeves and retaining nuts. The minimal modifications associated with the cannula hood of the present invention produce a lower cost device that is simple to use. Rather than going through several steps to attach an external sleeve to a syringe body with a retaining nut, a practitioner is able to slide the cannula hood onto the syringe body, where it is retained by a friction fit. Ease of use is a key advantage of the cannula hood of the present invention since speed and simplicity are of the essence in the context of ophthalmic viscosurgical procedures.

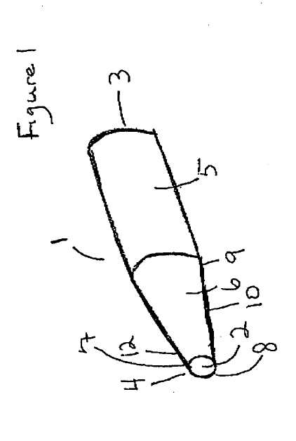

In the embodiment of the present invention shown in FIG. 1, the cannula hood 1 may form a lumen 2 and may form openings at its proximal 3 and distal 4 ends. Additionally, the cannula hood 1 may comprise a cylindrical portion 5 closest to its proximal end 3 and a tapered portion 6 closest to its distal end 4. The tapered portion 6 may comprise a proximal section 9, a mid section 10 and a distal section 12. The cylindrical portion 5 may be designed to fit over a syringe body and the tapered portion 6 may be designed to narrow at its apex 7 to provide an opening 8 that accommodates the diameter of a needle portion of a cannula assembly.

The cannula assembly may include a hub portion that facilitates coupling of the cannula assembly to the syringe body. The cannula hood 1 may be designed such that the proximal section 9 of the tapered portion 6 has a diameter greater than the diameter of the hub portion, the mid section 10 of the tapered portion 6 has a diameter that is equal to the diameter of the hub portion and the distal section 12 of the tapered portion 6 has a diameter that is less than the diameter of the hub portion. Thus, if the cannula assembly were to become disconnected from the syringe body during a viscosurgical procedure, the hub portion of the cannula assembly would make contact with the mid section 10 of the tapered portion 6 of the cannula hood 1. The cannula assembly would therefore be prevented from excessive movement towards the distal end 4 of the cannula hood 1 and, thus, would not become a projectile in the surgical field. Therefore, harmful disconnection of the cannula assembly from the syringe body would be prevented and damage to tissues in the surgical field would be ameliorated. Preferably, the tapered portion 6 may be designed to narrow in its diameter towards the distal end 4 of the cannula hood 1 such that, upon disconnection of the cannula assembly from the syringe body, movement of the cannula assembly towards the distal end 4 of the cannula hood 1 is largely or completely prevented. One skilled in the art will realize that this goal may be achieved by designing the tapered portion 6 such that the mid section 10 of the tapered portion 6 will be positioned immediately distal to the hub portion of the cannula assembly when the cannula hood 1 is attached to the syringe body.

The tapered portion 6 of the cannula hood 1 may be designed in the shape of a cone. The tapered portion 6 may also be designed such that its diameter narrows in a step-wise fashion when viewed from a proximal to distal direction. One skilled in the art will realize that many other tapered designs are possible and are encompassed by the description herein.

The cannula hood 1 may be constructed of plastic or rubber materials such as, but not in any way limited to, silicone, latex-free rubber, soft PVC, polycarbonate or acrylic. In one embodiment, the entirety of the cannula hood 1 may be constructed of a single material. In an alternative embodiment, the cylindrical portion 5 of the cannula hood 1 may be constructed of a softer material and the tapered portion 6 may be constructed of a firmer material. By way of example only, the cylindrical portion 5 may be constructed of silicone and the tapered portion 6 may be constructed of PVC. The cannula hood 1 may be constructed by injection molding or other means commonly known in the art. In an embodiment wherein the cannula hood 1 is constructed entirely of one material, a one-shot injection molding process may be used, although any other means of construction commonly known in the art may also be used. In an embodiment wherein the cannula hood 1 is constructed of two materials, a two-shot injection molding process may be used, although any other means of construction commonly known in the art may also be used. The cylindrical portion 5 of the cannula hood 1 may preferably be designed to form a friction fit with a syringe body. Other attachment means of the cannula hood 1 to the syringe body are possible, including, but not in any way limited to, adhesives and retaining devices. By way of example only, the proximal end 3 of the cylindrical portion 5 of the cannula hood 1 may be clipped onto a proximal end of a conventional Luer lock mechanism of a conventional syringe body. The cylindrical portion 5 of the cannula hood 1 may be designed in varying diameters so as to accommodate syringe bodies of varying diameters. The tapered portion 6 of the cannula hood 1 may narrow at its apex 7 to form an opening 8 that snugly or loosely accommodates the needle portion of a cannula assembly. The opening 8 may be designed with varying diameters so as to accommodate needles of varying diameters. When the tapered portion 6 of the cannula is formed of a softer material, it may flex to accommodate needles of varying diameters.

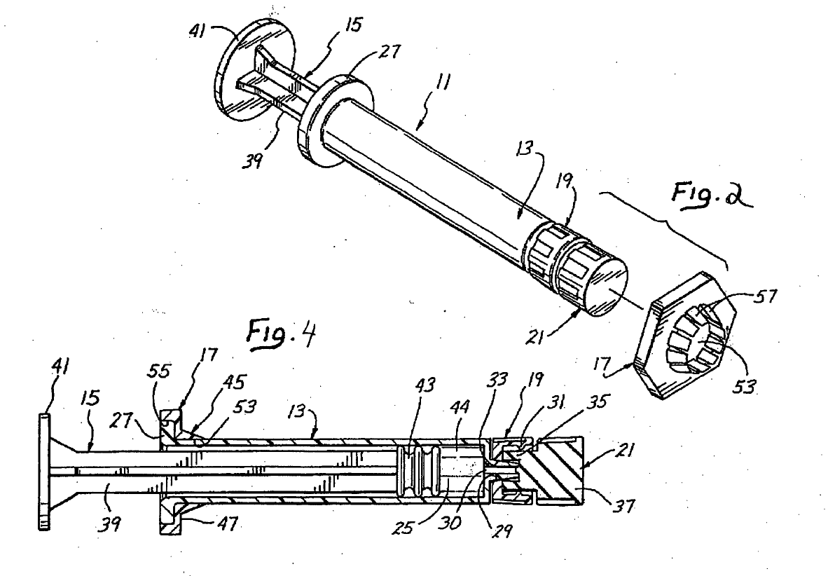

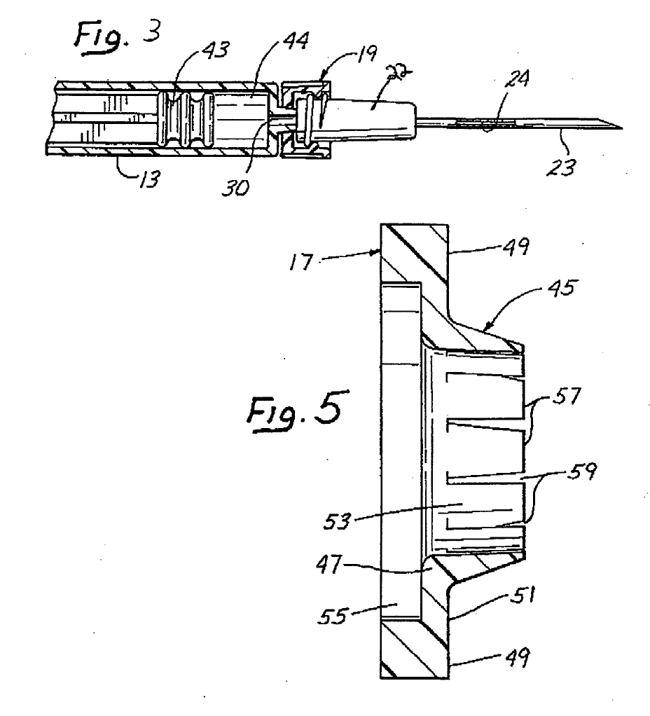

The cannula hood may adapt a single compartment syringe 11 such as, but not in any way limited to, that depicted in FIG. 2. The syringe 11 comprises a barrel 13, a plunger 15, an adapter 17, a connector in the form of a Luer lock fitting 19 and a tip cap 21. The syringe 11 may also be considered as including a cannula 23 (FIG. 3). The cannula 23 depicted in FIG. 3 has a small diameter passage 24 which may be, for example, about 0.008 inch to about 0.020 inch in diameter. The cannula 23 also has a hub 22, which is adapted for connection to the Luer lock fitting 19.

The barrel 13 may be of a common commercially available type which includes a cylindrical, longitudinal passage 25 extending through the barrel 13, and a circular flange 27 at the proximal end of the barrel 13. The distal end of the barrel 13 has an axially extending tip section 29 which is adapted for connection to the Luer lock fitting 19. The tip section 29 has an axial bore 30 extending through the tip section. Except for the flange 27 and the tip section 29, the barrel 13 is cylindrical. The barrel 13 is preferably integrally constructed of glass. The barrel 13 may alternatively be constructed of any material known in the art including, but not in any way limited to, plastic.

The Luer lock fitting 19 is also conventional and commercially available. As such, it includes an internally threaded socket 31 (FIG. 4) and an aperture 33 in its back wall for receiving the tip section 29 to which it is suitably attached as a mechanical interlock commonly used for Luer lock fittings. The Luer lock fitting 19 may also be constructed of polycarbonate.

The tip cap 21 is also a commercially available component and is provided for the purpose of sealing off the bore 30 in the tip section 29 to maintain sterility within the passage 25. The tip cap 21 is constructed of an elastomeric material and it includes an annular sealing skirt 35 and a knob 37 for use in installing and removing the tip cap.

The plunger 15 includes an elongated shank 39 which, in this embodiment has a plus or cross-shaped cross section and a pressure pad 41 at the proximal end of the shank 39. The plunger 15 may also be considered as including a conventional resilient piston 43 coupled to the distal end of the shank 39 and cooperable with the passage 25 to provide a pumping type function common to syringes. Thus, by moving the plunger 15 proximally, the piston 43 can draw a flowable material 44 into the passage 25 and by moving the plunger distally, the flowable material can be expelled from the passage 25 through the cannula 23 (FIG. 3). The pressure pad 41 is relatively large and may be, for example, about 0.74 inch in diameter. Although the plunger 15 may be constructed of various different materials, polycarbonate is preferred.

The adapter 17, which may also be constructed of polycarbonate, includes a resilient section 45 and a collar 47 which extends radially outwardly to form finger pads 49 on opposite sides of the adapter 17. More specifically, the finger pads 49 are simply diametrically opposed portions of an annular surface 51 which surrounds the resilient section 45.

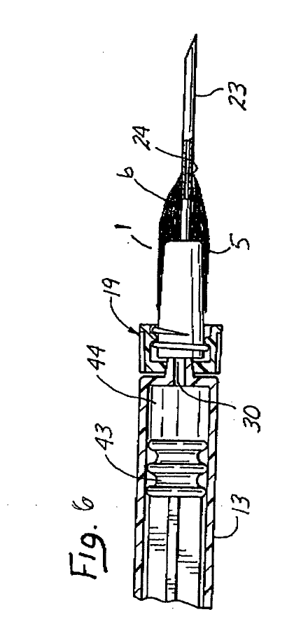

The adapter 17 also includes a bore or passage 53 extending axially completely through the adapter and a counterbore 55 of circular configuration sized and adapted to receive the flange 27. Although the resilient section 45 may be of various different constructions, in this embodiment it includes a plurality of resilient spring fingers 57 which are spaced circumferentially by axially extending slots 59. As shown in FIG. 5, the fingers 57 taper such that they become progressively thinner in their radial dimension as they extend distally.

As shown in FIG. 4, the passage 53 through the adapter 17 is adapted to receive the barrel 13 to mount the adapter 17 on the barrel 13. The adapter 17 can be positioned in engagement with the flange 27 of the barrel 13 with the counterbore 55 receiving the flange 27 as shown in FIG. 4. In this position, the adapter 17 extends radially beyond the flange 27 to provide the finger pads 49 which can be engaged by the index and middle fingers of the user when the thumb of the user presses on the thumb pad 41.

The resilient fingers 57 perform several important functions. For example, the adapter 17 can be assembled onto the barrel 13 by pushing the adapter over the tip cap 21 and the Luer lock fitting 19. In this regard, the Luer lock fitting 19 may be, and typically is, larger radially than the barrel 13. However, as clearly shown in FIGS. 2 and 4, the fitting 19 is not larger radially than the flange 27 and so more specifically the fitting 19 is larger radially than a main body portion of the barrel 13 which may include all of the barrel 13 except the flange 27. In this event, the resilient fingers 57 resiliently flex radially outwardly to allow the adapter to slide over the fitting 19 to mount the adapter on the barrel 13.

Once in position on the barrel 13, the fingers 57 resiliently grip the barrel to frictionally retard sliding movement of the adapter 17 along the barrel 13. In addition, the fingers 57 are engageable with the fitting 19 to positively resist sliding of the adapter 17 off of the connector 19.

Although the syringe 11 may be used with a variety of flowable materials 44, it is particularly adapted to be used with a sodium hyaluronate based material such as a sodium hyaluronate solution sold under the trademark Vitrax by Advanced Medical Optics, Inc. of Santa Ana, Calif. This material is injected into the posterior capsule of the eye during ophthalmic surgery in which the natural lens of the eye is being removed. Consequently, the syringe 11 may be sold with the passage 25 filled with the material 44 which may be a viscous flowable liquid, such as Vitrax or another hyaluronate based material.

Because the syringe is to be used in a medical context, it must be sterilized. Such sterilization is typically carried out using ethylene oxide gas. With the adapter 17 receiving the flange 27 in the counterbore 55, it is important to provide a flow path for sterilization gas to flow to the flange 27. Although this could be accomplished in different ways, in this embodiment, the flow path is provided by the slots 59 between the resilient fingers 57 and by mounting the collar 47 on the barrel 13 in a non-sealing relationship so that the sterilization gas can pass through the slots 59 and the unsealed interface between the collar 47 and the barrel 13.

In use of the syringe 11, the tip cap 21 is removed from the fitting 19 and the cannula 23 is attached to the fitting 19. The material 61 in the passage 25 can then be expelled through the small diameter passage 24 in the cannula 23 by moving the plunger 15 distally in a conventional manner. The larger area provided by the finger pads 49 and the pressure pad 41 facilitates the manual application of significant force to the plunger 15. Consequently, the plunger can be advanced to bring about movement or extrusion of the viscous material 61 through the small diameter passage 24 of the cannula 23.

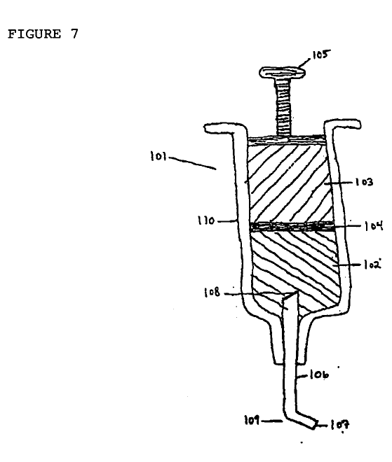

The cannula hood 1 may adapt the single compartment syringe 11 depicted in FIGS. 2-5 by sliding over the cannula 23 and forming a friction fit between the cylindrical portion 5 and the fitting 19. In the alternative, and as depicted in FIG. 6, the cannula hood 1 may be slid further in the proximal direction such that a friction fit is formed between the cylindrical portion 5 and the barrel 13. The cannula hood 1 may be designed such that the proximal section 9 of the tapered portion 6 has a diameter greater than the diameter of the hub portion 22, the mid section 10 of the tapered portion 6 has a diameter that is equal to the diameter of the hub portion 22 and the distal section 12 of the tapered portion 6 has a diameter that is less than the diameter of the hub portion 22. Thus, if the cannula 23 were to become disconnected from the syringe 11 during a viscosurgical procedure, the hub portion 22 of the cannula 23 would make contact with the mid section 10 of the tapered portion 6 of the cannula hood 1. The cannula 23 would therefore be prevented from excessive movement towards the distal end 4 of the cannula hood 1 and, thus, would not become a projectile in the surgical field. Therefore, harmful disconnection of the cannula 23 from the syringe 11 would be prevented and damage to tissues in the surgical field would be ameliorated.

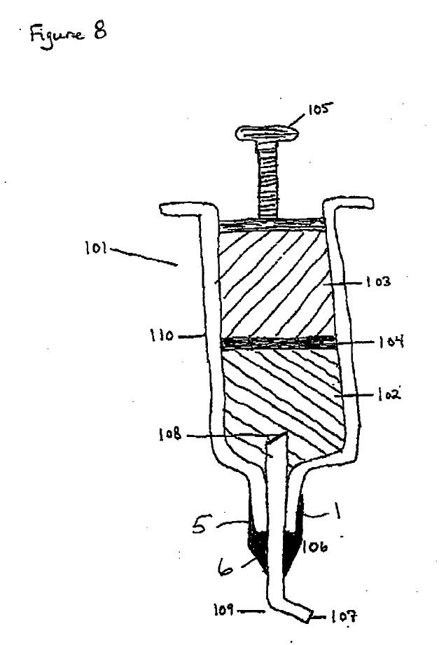

The cannula hood 1 may also adapt a bi-compartment syringe 101 such as, but not in any way limited to, that depicted in FIG. 7. Bi-compartment syringes adapted by the cannula hood 1 of the present invention may be largely similar in construction to the single compartment syringe 11 depicted in FIGS. 2-5, except for the addition of a second compartment that may contain an additional viscoelastic solution.

With reference to the bi-compartment syringe 101 depicted in FIG. 7, viscoelastic solutions are contained in separate compartments of the bi-compartment syringe 101 that are situated above each other, enclosed by a common housing 110. The compartments are optimally separated by a membrane or separation member 104. In this embodiment, the distal end of the bi-compartment syringe 101 may be a cannula 106 having a distal blunt end 107 that is external to the syringe body and a proximal sharp end 108 that is internal to the syringe body. Initially, the proximal sharp end 108 of the cannula 106 that is adjacent to the first viscoelastic solution 102 may be in fluid contact with the first viscoelastic solution 102 or may be separated from said first viscoelastic solution 102 by a membrane or other device. To dispense the first viscoelastic solution 102, the user applies pressure to a plunger 105 at the proximal end of the bi-compartment syringe 101. Said applied pressure forces the separation member 104 downward by fluid pressure, thus forcing the first viscoelastic solution 102 through the sharp proximal end 108 of the cannula 106 and expelling the first viscoelastic solution 102 from the distal blunt end 107 of the cannula 106. Upon application of additional pressure, the separation member 104 comes into contact with the sharp proximal end 108 of the cannula 106 and is then pierced. At this point, the cannula 106 is in fluid contact with the second viscoelastic solution 103. Continued pressure on the aforementioned plunger 105 serves to force the second viscoelastic solution 103 through the sharp proximal end 108 of the cannula 106 and expel the second viscoelastic solution 103 from the distal blunt end 107 of the cannula 106. One skilled in the art will realize that the cannula hood 1 of the present invention may also adapt a bi-compartment syringe wherein the compartments are arranged in a side-by-side design, rather than being arranged one on top of the other as described above.

As shown in FIG. 8, the cannula hood 1 may adapt the bi-compartment syringe 101 depicted in FIG. 7 by sliding over the cannula 106 and forming a friction fit between the cylindrical portion 5 and the housing 110. The cannula hood 1 may be designed such that the proximal section 9 of the tapered portion 6 has a diameter greater than the diameter of a hub portion of the cannula 106, the mid section 10 of the tapered portion 6 has a diameter that is equal to the diameter of the hub portion and the distal section 12 of the tapered portion 6 has a diameter that is less than the diameter of the hub portion. Thus, if the cannula 106 were to become disconnected from the syringe 101 during a viscosurgical procedure, the hub portion of the cannula 106 would make contact with the mid section 10 of the tapered portion 6 of the cannula hood 1. The cannula 106 would therefore be prevented from excessive movement towards the distal end 4 of the cannula hood 1 and, thus, would not become a projectile in the surgical field. Therefore, harmful disconnection of the cannula 106 from the syringe 101 would be prevented and damage to tissues in the surgical field would be ameliorated. One skilled in the art will realize that other multi-compartment devices, including those known in the art, may be used to separately introduce multiple viscoelastic solutions into the eye for concurrent use during ophthalmic surgery. Thus, one skilled in the art will realize that all such multi-compartment devices may be adapted by the cannula hood 1 of the present invention.

While the description above refers to particular embodiments of the present invention, it should be readily apparent to people of ordinary skill in the art that a number of modifications may be made without departing from the spirit thereof. The accompanying claims are intended to encompass such modifications as would fall within the true spirit and scope of the invention. The presently disclosed embodiments are, therefore, to be considered in all respects as illustrative and not restrictive. Accordingly, the scope of the invention is indicated by the appended claims rather than the foregoing description. All changes that come within the meaning and range of equivalency of the claims are intended to be embraced therein.

1. A cannula hood for a syringe, the cannula hood defining a lumen and openings at each of a proximal and distal end, the cannula hood comprising:
a cylindrical portion adjacent to the proximal end and configured to accommodate a syringe body; and
a tapered portion adjacent to the distal end, the tapered portion comprising a proximal section, a mid section and a distal section, the tapered portion further configured to accommodate a cannula assembly coupled to the syringe body.

2. The cannula hood of claim 1, wherein the diameter of the lumen tapers such that the diameter of the tapered lumen in the proximal section of the tapered portion is greater than or equal to the diameter of a hub portion of the cannula assembly and the diameter of the tapered lumen in the distal section of the tapered portion is less than the diameter of the hub portion.

3. The cannula hood of claim 1, wherein the cannula hood comprises one or more materials selected from the group consisting of plastic, rubber, silicone, latex-free rubber, PVC, polycarbonate and acrylic.

4. The cannula hood of claim 1, wherein both the cylindrical portion and the tapered portion comprise silicone.

5. The cannula hood of claim 1, wherein the cylindrical portion comprises silicone and the tapered portion comprises PVC.

6. The cannula hood of claim 1, wherein the cylindrical portion is retained on the syringe body by friction fit.

7. The cannula hood of claim 1, wherein the proximal end of the cylindrical portion is configured to clip onto a proximal end of a luer lock mechanism of the syringe body.

8. A system for performing ophthalmic surgery, the system comprising:
a syringe body;
a cannula assembly coupled to the syringe body; and
a cannula hood defining a lumen and openings at each of a proximal and distal end, the cannula hood comprising:
a cylindrical portion adjacent to the proximal end and configured to accommodate the syringe body; and
a tapered portion adjacent to the distal end, the tapered portion comprising a proximal section, a mid section and a distal section, the tapered portion further configured to accommodate a cannula assembly.

9. The system for performing ophthalmic surgery of claim 8, wherein the syringe body defines a single compartment.

10. The system for performing ophthalmic surgery of claim 8, wherein the syringe body defines two or more compartments.

11. The system for performing ophthalmic surgery of claim 8, wherein the diameter of the lumen tapers such that the diameter of the tapered lumen in the proximal section of the tapered portion is greater than or equal to the diameter of a hub portion of the cannula assembly and the diameter of the tapered lumen in the distal section of the tapered portion is less than the diameter of the hub portion.

12. The system for performing ophthalmic surgery of claim 8, wherein the cannula hood comprises one or more materials selected from the group consisting of plastic, rubber, silicone, latex-free rubber, PVC, polycarbonate and acrylic.

13. The system for performing ophthalmic surgery of claim 8, wherein both the cylindrical portion and the tapered portion comprise silicone.

14. The system for performing ophthalmic surgery of claim 8, wherein the cylindrical portion comprises silicone and the tapered portion comprises PVC.

15. The system for performing ophthalmic surgery of claim 8, wherein the cylindrical portion is retained on the syringe body by friction fit.

16. The system for performing ophthalmic surgery of claim 8, wherein the proximal end of the cylindrical portion is configured to clip onto a proximal end of a luer lock mechanism of the syringe body.

17. A method of performing ophthalmic surgery comprising:
attaching a cannula hood to a syringe apparatus, the syringe apparatus comprising a syringe body and a cannula assembly, the cannula hood defining a lumen and openings at each of a proximal and distal end, the cannula hood comprising:
a cylindrical portion adjacent to the proximal end and configured to accommodate the syringe body; and
a tapered portion adjacent to the distal end, the tapered portion comprising a proximal section, a mid section and a distal section, the tapered portion further configured to accommodate a cannula assembly;
forming an incision in an eye; and
injecting one or more viscoelastic agents into the eye.

18. The method of performing ophthalmic surgery of claim 17, wherein the syringe body defines one compartment.

19. The method of performing ophthalmic surgery of claim 17, wherein the syringe body defines two or more compartments.

20. The method of performing ophthalmic surgery of claim 17, wherein the diameter of the lumen tapers such that the diameter of the tapered lumen in the proximal section of the tapered portion is greater than or equal to the diameter of a hub portion and the diameter of the tapered lumen in the distal section of the tapered portion is less than the diameter of the hub portion.

21. The method of performing ophthalmic surgery of claim 17, wherein the cannula hood comprises one or more materials selected from the group consisting of plastic, rubber, silicone, latex-free rubber, PVC, polycarbonate and acrylic.

22. The method of performing ophthalmic surgery of claim 17, wherein both the cylindrical portion and the tapered portion comprise silicone.

23. The method of performing ophthalmic surgery of claim 17, wherein the cylindrical portion comprises silicone and the tapered portion comprises PVC.

24. The method of performing ophthalmic surgery of claim 17, wherein the cylindrical portion is retained on the syringe body by friction fit.

25. The method of performing ophthalmic surgery of claim 17, wherein the proximal end of the cylindrical portion is configured to clip onto a proximal end of a luer lock mechanism of the syringe body.

26. The method of performing ophthalmic surgery of claim 17, wherein the syringe body is pre-filled with the one or more viscoelastic agents.

27. A method of preventing disconnection of a cannula assembly from a syringe body during ophthalmic surgery comprising:
attaching a cannula hood to a syringe apparatus, the syringe apparatus comprising a syringe body and a cannula assembly, the cannula hood defining a lumen and openings at each of a proximal and distal end, the cannula hood comprising:
a cylindrical portion adjacent to the proximal end and configured to accommodate the syringe body; and
a tapered portion adjacent to the distal end, the tapered portion comprising a proximal section, a mid section and a distal section, the tapered portion configured to accommodate a cannula assembly.

28. The method of preventing disconnection of a cannula assembly from a syringe body of claim 27, wherein the syringe body defines a single compartment.

29. The method of preventing disconnection of a cannula assembly from a syringe body of claim 27, wherein the syringe body defines two or more compartments.

30. The method of preventing disconnection of a cannula assembly from a syringe body of claim 27, wherein the diameter of the lumen tapers such that the diameter of the tapered lumen in the proximal section of the tapered portion is greater than or equal to the diameter of a hub portion of the cannula assembly and the diameter of the lumen in the distal section of the tapered portion is less than the diameter of the hub portion.

31. The method of preventing disconnection of a cannula assembly from a syringe body of claim 27, wherein the cannula hood comprises one or more materials selected from the group consisting of plastic, rubber, silicone, latex-free rubber, PVC, polycarbonate and acrylic.

32. The method of preventing disconnection of a cannula assembly from a syringe body of claim 27, wherein both the cylindrical portion and the tapered portion comprise silicone.

33. The method of preventing disconnection of a cannula assembly from a syringe body of claim 27, wherein the cylindrical portion comprises silicone and the tapered portion comprises PVC.

34. The method of preventing disconnection of a cannula assembly from a syringe body of claim 27, wherein the cylindrical portion is retained on the syringe body by friction fit.

35. The method of preventing disconnection of a cannula assembly from a syringe body of claim 27, wherein the proximal end of the cylindrical portion is configured to clip onto a proximal end of a luer lock mechanism of the syringe body.
